(19) United States
(12) Patent Application Publication
(10) Pub. No.: US 2026/0335202 A1
(43) Pub. Date: Oct. 29, 2026

(54) ORAL CARE COMPOSITION AND METHOD FOR PREPARING ORAL CARE COMPOSITION

(71) Applicant: LG H&H Co., LTD., Seoul (KR)

(72) Inventors: Ji Young KIM, Seoul (KR); Seongwoo BAK, Seoul (KR); Brian CHUNG, Seoul (KR); Wonho HA, Seoul (KR)

(73) Assignee: LG H&H Co., LTD., Seoul (KR)

(21) Appl. No.: 19/099,176

(22) PCT Filed: Jul. 28, 2023
(86) PCT No.: PCT/KR2023/011055

§ 371 Date: Jan. 28, 2025

(30) Foreign Application Priority Data
Jul. 29, 2022 (KR) 10-2022-0094833
Jul. 27, 2023 (KR) 10-2023-0098513

(51) Int. Cl. A61K 8/27; A61K 8/20; A61K 8/21; A61Q 11/00

(52) U.S. Cl. CPC A61K 8/27; A61K 8/20; A61K 8/21; A61Q 11/00

(57) ABSTRACT

Provided are an oral care composition comprising stannous fluoride which is stabilized in the form of stannous gluconate, and a method of preparing the oral care composition.

### TECHNICAL FIELD

The present disclosure relates to an oral care composition and a method for preparing oral care composition.

### BACKGROUND ART

With the growing interest in personal hygiene, including oral health, the number of consumers is increasing who want to achieve more than just brushing their teeth with a toothpaste or mouthwash daily used, rather than using separate products for oral health. Meanwhile, active ingredients that help oral health are highly effective when used alone, but there has been a problem that they are less effective when used with common toothpaste ingredients or their long-term storage stability is poor.

Toothpaste including stannous fluoride has antigingivitis, anticavity, and desensitizer effects. However, stannous fluoride has a problem that it is easily hydrolyzed and oxidized in water and becomes tin ion (Sn⁴⁺) to lose its activity. In addition, stannous fluoride toothpaste may cause staining by combining with sulfides in the oral cavity. In order to obtain a desired effect by stabilizing stannous fluoride, it is preferably applied in the form of low-water toothpaste with a water content below a predetermined level or in the form of anhydrous toothpaste. However, low-water or anhydrous toothpastes have a problem of low user preference due to the bitter taste and burning sensation unique to polyols.

Accordingly, there is a need for a combination of compositions capable of stabilizing stannous fluoride while securing the gingivitis, anticavity, and desensitizer effects of stannous fluoride.

### DISCLOSURE

### Technical Problem

An object of the present disclosure is to secure the gingivitis, anticavity, and desensitizer effects of stannous fluoride by stabilizing stannous fluoride.

### Technical Solution

According to an embodiment of the present disclosure, there is provided an oral care composition comprising a zinc compound that is dissociated in an aqueous solvent to provide zinc ions; and a stannous compound of which at least part is provided in the form of stannous gluconate, wherein the stannous compound comprises stannous chloride (SnCl₂) and stannous fluoride (SnF₂).

According to an embodiment of the present disclosure, there is provided the oral care composition, wherein the zinc compound has a solubility of more than 0.6% and less than 69% in the aqueous solvent.

According to an embodiment of the present disclosure, there is provided the oral care composition, wherein the zinc compound is at least one selected from zinc citrate, zinc lactate, and zinc acetate.

According to an embodiment of the present disclosure, there is provided the oral care composition, further comprising a pH adjuster and an abrasive.

According to an embodiment of the present disclosure, there is provided the oral care composition, of which pH is 6 to 10.

According to an embodiment of the present disclosure, there is provided a method of preparing the oral care composition, the method comprising a first step of forming stannous gluconate by introducing stannous chloride (SnCl₂) to a solution in which sodium gluconate is dissolved; a second step of stabilizing stannous fluoride (SnF₂) in the form of stannous gluconate by adding stannous fluoride (SnF₂) to the mixed solution comprising stannous gluconate; and a third step of adding the solution comprising stannous fluoride of the second step to a zinc solution comprising dissociated zinc ions.

According to an embodiment of the present disclosure, there is provided the method of preparing the oral care composition, wherein the pH adjuster is further added to the mixed solution after the third step.

According to an embodiment of the present disclosure, there is provided the method of preparing the oral care composition, wherein the zinc compound solution comprising the zinc ion is prepared by dissolving a zinc compound having a solubility of more than 0.6% and less than 69% in an aqueous solvent.

### Advantageous Effects

According to the present disclosure, it is possible to secure antigingivitis, anticavity, and desensitizer effects of stannous fluoride by stabilizing stannous fluoride.

Further, according to the present disclosure, since acidic conditions are not required to stabilize stannous fluoride, it is possible to prevent adverse effects such as caries, mucosal irritation, etc. which may be exhibited in the oral cavity by acidic compositions.

### DETAILED DESCRIPTION OF PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

According to an embodiment of the present disclosure, there is provided an oral care composition comprising a zinc compound that is dissociated in an aqueous solvent to provide zinc ions; and a stannous compound that is stabilized in the form of stannous gluconate, wherein the stannous compound comprises stannous chloride (SnCl₂) and stannous fluoride (SnF₂).

The oral care composition may be a composition, such as toothpaste, gargle, etc., which is applied to the oral cavity to provide useful effects for a user, such as antigingivitis, anticavity, and desensitizer effects, etc. There are no limitations on the phase or provision form of the oral care composition. For example, the oral care composition may be provided in various forms, such as toothpaste, gargle, etc.

The oral care composition comprises stabilized stannous fluoride. The stannous fluoride may have a composition of SnFx. For example, the stannous fluoride may a have composition of SnF₂. The stannous fluoride may exhibit antigingivitis, anticavity, and desensitizer effects in the oral cavity.

The stannous fluoride is provided in a stabilized form. The stabilized stannous fluoride may mean a form in which a divalent stannous ion (Sn²⁺) dissociated from a compound in the form of SnF₂ maintains a divalent charge without being oxidized to a tetravalent stannous ion (Sn⁴⁺).

The stannous fluoride is an active ingredient that exhibits antigingivitis, anticavity, and desensitizer effects in the oral cavity, and its content may be determined by considering the intensity of the effects of the oral care composition, the content of other elements added for stabilization, and the form of storage and use.

The above-mentioned stabilized stannous fluoride is provided in the form of stannous gluconate (Sn gluconate). Stannous gluconate may mean a complex in which gluconate is coordinated to a divalent stannous ion (Sn²⁺). The divalent stannous ion (Sn²⁺) provided in the form of stannous gluconate may maintain a divalent charge without being oxidized to a tetravalent stannous ion (Sn⁴⁺).

The above-mentioned stannous gluconate (Sn gluconate) may be provided from a gluconate compound, for example, sodium gluconate. The sodium gluconate may be dissolved in a solvent to provide gluconate. The gluconate may form a complex ion by coordinating with divalent stannous ion at more than two sites.

The sodium gluconate is a component that forms stannous gluconate together with stannous fluoride and/or stannous chloride, and thus the addition amount may be determined by considering the contents of stannous fluoride and stannous chloride comprised in the oral care composition.

In addition, stannous chloride (SnCl₂) is generally more easily oxidized than stannous fluoride (SnF₂). It is known that chloride ion may act as a stronger oxidizing agent than fluoride ion. This means that stannous chloride (SnCl₂) may be more easily oxidized to stannous chloride (SnCl₄), of which oxidation state is +4, in the presence of oxygen or other oxidizing species. In contrast, stannous fluoride has a less tendency to be oxidized and tends to more effectively maintain its oxidation state of +2. Therefore, stannous chloride (SnCl₂) provided in the oral care composition may be dissociated in the oral care composition, and as a sacrificial ion, may stabilize stannous fluoride. Therefore, the fact that stannous chloride (SnCl₂) acts as a sacrificial ion may mean that even when an oxidation reaction occurs in the oral care composition, stannous chloride is oxidized instead of stannous fluoride, thereby preventing the oxidation of stannous fluoride and stabilizing stannous fluoride.

As described above, stannous chloride is converted into the form of stannous gluconate and may function as a sacrificial ion that is oxidized instead of stannous fluoride. Therefore, the content may be determined by considering factors such as the amount of stannous fluoride comprised in the oral care composition and the amount of zinc ion, which is another element that stabilizes stannous fluoride.

As mentioned above, sodium gluconate is a chelating agent that forms a stable complex with metal ions comprising stannous ion. In the presence of water and oxygen, stannous chloride and stannous fluoride may decompose and lose their effectiveness over time. However, addition of sodium gluconate to tin-comprising products may improve the stability of tin. Sodium gluconate forms a complex with stannous ion, preventing the precipitation and oxidation of stannous chloride and stannous fluoride, protecting their activity, and ultimately, may help to maintain the stability and efficacy of stannous fluoride in toothpaste throughout the expiration date.

A zinc compound that is dissociated in an aqueous solvent to provide zinc ions is provided in the oral care composition. The zinc compound is in the form of a salt, and exists in the dissociated form of zinc ions and negative ions in the oral care composition.

The dissociated zinc ions (Zn²⁺) have an excellent reducing effect, and are effective in preventing the oxidation of divalent stannous ions (Sn²⁺). Therefore, the dissociated zinc ions may further secure the stabilization of stannous fluoride. The dissociated zinc ions are introduced not in the form of a zinc salt, but in a dissociated state, and thus it may immediately act on the stabilized stannous fluoride to provide a reducing effect. Accordingly, the stabilization of stannous fluoride may be immediately secured. For example, even though zinc citrate salt itself is not a reducing agent, the dissociated zinc ion may function as a reducing agent. Therefore, there may be differences in the function of preventing oxidation of stannous ion, depending on whether it is applied to the prescription in a salt state or as a dissociated zinc ion.

In addition, citrate, lactate, or acetate comprised in zinc citrate, zinc lactate, or zinc acetate may act as a chelating agent for stannous ion, like sodium gluconate, and thus may contribute to the stabilization of stannous ion.

The dissociated zinc ions may be provided from the zinc compound having a solubility of more than 0.6% and less than 69% in an aqueous solvent (water).

The zinc compound described above may be a zinc salt, such as zinc citrate, zinc lactate, zinc acetate, etc., which has an appropriate solubility in an aqueous solution to relatively easily dissociate zinc ions. In this regard, it is difficult to use insoluble zinc salts, such as zinc oxide, zinc pyrophosphate, zinc laurate, zinc phosphate, zinc polyphosphate, zinc stearate, etc., as the zinc compound in the present disclosure. In addition, when the solubility of zinc ion for water is too high, it is dissolved to 100% in even a small amount of water, which will significantly affect the taste of a product due to the bitter taste unique to zinc ion. In addition, zinc salts with very high solubility for water, for example, zinc chloride and zinc nitrate, may easily form an insoluble complex, zinc fluoride, when added to a solution in which fluoride is dissolved, which may reduce the activity of fluoride, leading to deterioration of the anticavity effect of fluoride.

As described above, the oral care composition according to one embodiment of the present disclosure provides stannous fluoride in the form of stannous gluconate in the mixed composition, and the divalent stannous ion (Sn²⁺) in the stannous fluoride may be stabilized without being additionally oxidized, due to the sacrificial ion effect of stannous chloride provided in the composition and the reduction effect of zinc ions provided from the zinc compound. However, the specific mechanism is not limited to the above-described description.

In particular, according to one embodiment of the present disclosure, stannous fluoride may be provided by stabilizing in an environment other than an acidic condition. For example, according to the prior art, in order to stabilize stannous fluoride by sodium gluconate and/or stannous chloride, an acidic condition of pH 5 or lower was required. The acidic condition may cause caries in the oral cavity and may irritate the oral cavity. In addition, since polyphosphonate which is used to create the acidic condition is not a raw material generally used in toothpaste, there is a stability problem.

The oral care composition may further comprise a pH adjuster. The pH adjuster is comprised in the oral care composition comprising the dissociated zinc ions and stabilized stannous fluoride, thereby may adjust the oral care composition to a desired pH range. The pH adjuster may be sodium hydroxide, sodium citrate, sodium phosphate, sodium succinate, sodium tartrate, etc., and may be appropriately selected depending on the pH range to be adjusted.

The pH of the oral care composition may be 6 to 10. As described, the oral care composition according to one embodiment of the present disclosure may have a pH in a neutral or alkaline range. An acidic oral care composition with a pH of 5 or less may cause caries or oral irritation when used, but the oral care composition according to one embodiment of the present disclosure does not have the above-mentioned problem because it is neutral or alkaline.

The divalent stannous ion (Sn²⁺) comprised in stannous fluoride is not further oxidized even under neutral or basic conditions, not conditions, acidic and may provide antigingivitis, anticavity, and desensitizer effects in the divalent ionic state. Furthermore, in oral care the composition, divalent stannous ions (Sn²⁺) are stabilized by the dissociated zinc ions described above even in an environment other than acidic conditions, and thus there is no concern that the acidic composition will irritate a user's mouth.

The oral care composition may further comprise an abrasive, a humectant, a flavoring agent, a sweetening agent, a preservative, a binder, a foaming agent, a whitening agent, etc.

The abrasive is a substance that has the function of removing dental plaque in the oral cavity, and may be used to increase the removal efficiency of dental plaque and to remove hard foreign substances, etc., and may exhibit a Mohs hardness of about 3 to about 6.

The humectant means a substance that acts to reduce friction between two sides sliding in contact with each other, and the humectant functions to lubricate, thereby playing a role in preventing abrasion of a piston by a raw material (solids such as an abrasive, etc.) showing wear properties, which is comprised in the toothpaste composition of the present disclosure. The humectant may comprise, for example, any one selected from the group consisting of polyethylene glycol, glycerol, propylene glycol, ethylene glycol, polypropylene glycol, and mixtures thereof, but is not limited thereto. Preferably, it may comprise any one selected from the group consisting of polyethylene glycol 200 to 600, glycerol, propylene glycol, ethylene glycol, polypropylene glycol, and mixtures thereof, Preferably, as a liquid humectant, petroleum oil, animal and vegetable oil, synthetic lubricating oil, etc. may be used, and most preferably, in terms of stability and excellent usability of the composition, a liquid polyol may be used. Otherwise, the humectant is not limited to the liquid polyol, and it may comprise a high-molecular-weight polyol, like polyethylene glycol or polypropylene glycol which has a polymeric form, which may be present in a solid phase at a room temperature by intramolecular interaction of polymers over a certain molecular weight, but may be liquefied by controlling the preparation temperature and may be maintained in a stable state when manufactured.

The flavoring agent remains in the oral cavity and allows to maintain a sense of freshness by continually emitting fragrances. As the flavoring agent, mint like peppermint, spearmint, etc., wintergreen, methyl salicylate, eugenol, melon, strawberry, orange, vanillin, etc. may be used.

The sweetening agent may be added to overcome the basic taste which may be possessed by the formulation. The sweetening agent may play a role in sustaining occurrence of saliva by providing the taste continuously while remaining in the oral cavity. As the sweetening agent, saccharin, sucralose, sugar, xylitol; sorbitol, lactose, mannitol, maltitol, erythritol, aspartame, taurine, saccharin salt, D-tryptophan, etc. may be used alone or in a mixture of two or more thereof.

As the preservative, benzoic acid, methyl paraben, propyl paraben, sodium benzoate, etc. may be used.

As the foaming agent, an anionic, amphoteric, and non-ionic surfactant such as sodium alkyl sulfate, sodium lauryl sulfate, alkyl sarcosinate, lauroyl sarcosinate, cocoyl glutamate sodium salt, myristoyl glutamate sodium salt, cocamidopropyl betaine, sucrose fatty acid ester, sorbitan fatty acid ester, a polyoxyethylene-polyoxypropylene block copolymer (poloxamer), etc. may be used alone or in a mixture of two or more thereof.

As the whitening agent, titanium oxide may be used.

The content and type of the above-described abrasive, humectant, flavoring agent, sweetening agent, preservative, binder, foaming agent, and whitening agent may be determined by considering the effect on the dissociated zinc ion, stannous fluoride, sodium gluconate, and stannous chloride provided in the oral care composition.

Hereinbelow, a method of preparing the oral care composition according to one embodiment of the present disclosure will be described.

The method of preparing the oral care composition according to one embodiment of the present disclosure comprises a first step of forming stannous gluconate by introducing stannous chloride (SnCl₂) to a solution in which sodium gluconate is dissolved; a second step of stabilizing stannous fluoride (SnF₂) in the form of stannous gluconate by adding stannous fluoride (SnF₂) to the mixed solution comprising stannous gluconate; and a third step of adding the solution comprising stannous fluoride of the second step to a zinc solution comprising dissociated zinc ions.

In the method of preparing the oral care composition of the present disclosure, the first to third steps may be sequentially performed. In other words, the steps of dissolving sodium gluconate, adding stannous chloride, adding stannous fluoride, and adding the zinc compound solution comprising zinc ions may be sequentially performed. In particular, when the steps of dissolving sodium gluconate, adding stannous chloride, adding stannous fluoride, and adding the zinc compound solution comprising zinc ions are sequentially performed, the effect of stabilizing divalent stannous ion (Sn²⁺) is high, thereby may increase the concentration of divalent stannous ion (Sn²⁺) in the oral care composition, as compared to the case where the steps are performed at the same time.

Hereinafter, regarding each step of the method of preparing the oral care composition, performed is the first step of forming stannous gluconate by introducing stannous chloride (SnCl₂) to a solution in which sodium gluconate is dissolved.

To perform the first step, the solution in which sodium gluconate is dissolved is first prepared. Sodium gluconate is dissolved in an aqueous solvent, such as water, and may exist after being dissociated into sodium ion and gluconate.

Next, stannous chloride (SnCl₂) is introduced to the solution, in which sodium gluconate is dissociated into sodium ion and gluconate, to form stannous gluconate. Stannous chloride (SnCl₂) may be mixed with the dissociated sodium gluconate before stannous fluoride (SnF₂) to form stannous gluconate (Sn gluconate). In addition, stannous chloride as a sacrificial ion may prevent additional oxidation of stannous fluoride to be added later. In particular, since stannous chloride is mixed with sodium gluconate before stannous fluoride, when stannous fluoride is added in the second step, stannous chloride and sodium gluconate may be in a state where they are sufficiently dissolved in the solution. Therefore, when stannous fluoride is added later, the sacrificial ion effect of stannous chloride and the stabilization of divalent stannous ion (Sn²⁺) due to the formation of stannous gluconate may occur immediately.

In the second step which is performed after the first step, stannous fluoride (SnF₂) is added to the mixed solution comprising stannous gluconate to stabilize stannous fluoride (SnF₂) in the form of stannous gluconate.

In the second step, stannous fluoride (SnF₂) reacts with the gluconate provided in the mixed solution and is stabilized in the form of stannous gluconate. In addition, as previously explained, since stannous chloride exists in the mixed solution, stannous chloride acts as a sacrificial ion to prevent the oxidation of stannous fluoride.

Next, performed is the third step of adding the solution comprising stannous fluoride of the second step to the zinc solution comprising dissociated zinc ions.

Before performing the third step, a step of dissociating the zinc compound by dissolving the zinc compound in an aqueous solvent to provide zinc ion and anion may be performed. In one embodiment of the present disclosure, instead of providing the zinc compound in the form of a salt for the mixed solution, the zinc compound, in the form of a solution in which zinc ions are dissociated, is mixed with the mixed solution, and therefore, the reduction of the divalent stannous ion by the zinc ions may immediately occur.

After the third step, a pH adjuster may be further added to the mixed solution. Therefore, since the pH adjuster is added to the oral care composition, after the third step is performed and the divalent stannous ion of the stannous fluoride is stabilized, the divalent stannous ions may be provided in a stabilized form into the oral care composition, regardless of the pH change, even when an acidic condition is changed to a neutral or basic condition.

In order to perform the first to third steps, additional treatments such as stirring, heating, etc. may be performed. In addition, a heating process may be further performed to increase the temperature of the mixed solution for increasing solubility of the compound in the solution, as needed.

As described above, with regard method of preparing the oral care composition according to one embodiment of the present disclosure, the steps of dissolving sodium gluconate, adding stannous chloride, adding stannous fluoride, and adding the zinc compound solution comprising zinc ions are sequentially performed, thereby achieving a high stabilization effect of divalent t stannous ion (Sn²⁺), and accordingly, may increase the concentration of divalent stannous ion (Sn²⁺) in the oral care composition.

### MODE FOR INVENTION

As above, an oral care composition and a preparation method thereof according to one embodiment of the present disclosure have been examined. Hereinafter, the excellent effects of the oral care composition and the preparation method thereof according to the present disclosure will be examined through Examples and Comparative Examples.

### Experimental Example 1. Verification of Sn²⁺ Ion Stabilization Effect According to Order of Component Input

A preparation method of Example 1 is as follows. In detail, in a main tank, a polymer binder (carrageenan, xanthan gum, sodium carboxymethyl cellulose, carbomer, etc.) was pre-dispersed in a humectant (sorbitol, polyethylene glycol, glycerin, etc.), and then a salt component (saccharin, etc.) dissolved or dispersed in purified water was added to hydrate the polymer under stirring. A stabilizer, zinc ion (zinc citrate) was added thereto and dispersed and dissolved.

Next, water was added to a separate sub tank, and a stannous fluoride stabilizer (stannous chloride and sodium gluconate) and stannous fluoride were added to disperse and dissolve, and then put into the main tank.

Thereafter, silica (polishing silica, thickening silica) and a pH adjuster were dispersed and mixed in the main tank and stirred for 20 minutes. Next, a foaming agent (SLS, etc.) was added to the main tank and the mixture was stirred for 20 minutes under vacuum. After adding fragrance to the stirred mixture, a toothpaste composition was prepared with stirring under vacuum for 20 minutes. However, the above-described preparation method is exemplary, and a detailed method of performing the process may be changed as long as the preparation method according to the present disclosure described above is followed, and thus the toothpaste composition of the present disclosure is not limited to the above-described preparation method.

The compositions of Comparative Examples 1 to 3 were prepared in the similar manner as Example 1, but differences were made in the order and method of adding stannous fluoride (SnF₂), stannous chloride (SnCl₂), sodium gluconate, zinc citrate, and pH adjuster. The differences in the process between Comparative Examples 1 to 3 and Example 1 are as shown in Table 1 below.

For all of Example and Comparative Examples, stannous fluoride (SnF₂) was added in an amount of 0.454 wt % with respect to the total weight of the mixed composition, and stannous chloride (SnCl₂) was added in an amount of 0.540 wt % with respect to the total weight of the mixed composition.

TABLE 1

| No. | Process |
|---|---|
| Comparative | 1) SnF₂, SnCl₂, sodium gluconate, Zn citrate, and NaOH were |
| Example 1 | added to the sub tank at the same time, and dispersed, and then |
| | the mixed solution was introduced into the main tank. |
| Comparative | 1) SnF₂, SnCl₂, sodium gluconate, and Zn citrate were added to |
| Example 2 | the sub tank at the same time, and dispersed, and then the mixed |
| | solution was introduced into the main tank. |
| | 2) Then, NaOH and silica were introduced into the main tank. |
| Comparative | 1) Sodium gluconate, SnCl₂, and SnF₂ were sequentially |
| Example 3 | dispersed in the sub tank, and then the mixed solution was |
| | introduced into the main tank. |
| | 2) Then, zinc citrate was introduced, together with NaOH and |
| | silica, into the main tank. |
| Example 1 | 1) Zinc citrate was dispersed in the main tank. |
| | 2) Sodium gluconate, SnCl₂, and SnF₂ were sequentially |
| | introduced and dissolved using the sub tank, and then |
| | introduced into the main tank. |
| | 3) Then, NaOH and silica were introduced into the main tank. |

In this Experimental Example, the stabilization effect of divalent stannous ion (Sn²⁺) in the oral care composition (toothpaste) was examined by changing the order of introducing stannous fluoride (SnE₂), stannous chloride (SnCl₂), sodium gluconate, zinc citrate, and NaOH. The concentration of divalent stannous ion in the toothpaste was measured using indirect iodometric titration. An electrode used for performing the indirect iodometric titration was a redox electrode (DMi 140-SC redox pH electrode, Mettler Toledo, Switzerland), together with an automatic titrator (G20 auto titrator, Mettler Toledo, Switzerland). The standard calibration curve of divalent stannous ion was determined with stannous chloride standard solutions (0.05 M, 0.25 M, 0.1 M, 0.05 M). As a titrant, a 0,005 N sodium thiosulfate solution was used. The reaction scheme is as follows. The solutions used for titration were a 0.01 N KIO₃ solution, a 10% KI solution, and 1 N HCl.

Sn²⁺+I₂(known excess)→Sn⁴⁺+21⁻+I₂(excess unreacted iodine) (1)

In the reaction scheme of (1), I₂ (excess unreacted) is prepared in-situ in the following reaction (2).

IO₃⁻+5I⁻+6H⁺→3I₂+3H₂O (2)

In the reaction scheme of (1), I₂ (excess unreacted) is quantified with a titration solution (sodium thiosulfate, 2S₂O₃²⁻) as in the following reaction scheme.

I₂ (excess unreacted)+2S₂O₃²⁻→S₄O₆²⁻+21⁻ (3)

According to the above reaction scheme, the divalent stannous ions reacted with 0.01N KIO₃ solution may be calculated by the following relationship (4):

1mL of 0.01N KIO₃=0.594mg Sn²⁺ (4)

For the toothpastes prepared according to Example and Comparative Examples, the concentration of Sn²⁺ was examined 8 weeks after preparation. The concentration of Sn²⁺ after 8 weeks was described together with the ratio (%) relative to the theoretical content of Sn²⁺ ion initially added. The theoretical content value of Sn²⁺ ion added to the toothpaste was 0.628 wt %. The theoretical content of Sn²⁺ ion means the ion concentration when Sn²⁺ is not completely oxidized but maintains the divalent ionic form after preparing the toothpaste composition,

TABLE 2

| | Sn²⁺ |
|---|---|
| | concentration |
| | (wt %) in |
| | toothpaste |
| | composition |
| | 8 weeks after |
| No. | preparation |
| Comparative Example 1 | 0.235 ± 0.043 (37%) |
| Comparative Example 2 | 0.270 ± 0.019 (43%) |
| Comparative Example 3 | 0.203 ± 0.093 (32%) |
| Example 1 | 0.450 ± 0.007 (72%) |

As a result of preparing the oral care compositions according to Example and Comparative Examples, the toothpaste of Example 1, in which zinc citrate was introduced into the main tank, and sodium gluconate, stannous chloride, and stannous fluoride were sequentially dissolved in water in the separate sub tank, and this solution was introduced into the main tank in which zinc citrate was dissolved, and then NaOH and silica were introduced, showed the highest concentration of divalent stannous ions in the toothpaste. This means that divalent stannous ions were stabilized in the toothpaste, and accordingly, the concentration of divalent stannous ions in the toothpaste remained high even after 8 weeks. In contrast, in Comparative Example 1, in which stannous fluoride, stannous chloride, sodium gluconate, zinc citrate, and NaOH were simultaneously added in a salt form without being dissolved in water, the divalent stannous ion (Sn²⁺) was not sufficiently stabilized, and thus the concentration of divalent stannous ion in the toothpaste was relatively low (37%) 8 weeks after preparation.

Further, in Comparative Example 2, stannous fluoride, stannous chloride, sodium gluconate, and zinc citrate were simultaneously added in a salt form without being dissolved in water, and subsequently, NaOH was introduced. In this case, since pH increased while the divalent stannous ion (Sn²⁺) was rather stabilized before adding NaOH, the concentration of divalent stannous ion in the toothpaste was high (43%), as compared to Comparative Example 1.

In Comparative Example 3, sodium gluconate, stannous chloride, and stannous fluoride were sequentially dissolved in water and introduced, and subsequently, zinc citrate and NaOH were introduced. In this case, zinc citrate and NaOH were subsequently introduced, and the pH of the toothpaste increased due to the introduction of NaOH before sufficient stabilization of the divalent stannous ion (Sn²⁺) by the zinc ion, which led to some oxidation of the divalent stannous ion (Sn²⁺). Accordingly, the concentration of divalent stannous ion in the toothpaste was found to be relatively low (32%) 8 weeks after preparation.

### Experimental Example 2. Verification of Sn²⁺ Ion Stabilization Effect According to Presence or Absence of Stabilizer

In this Experimental Example, all other conditions were kept the same, particularly, the condition of dispersing zinc citrate first in the main tank was the same, and the results according to combinations of other components were compared in a state where the zinc ion was stabilized:

In detail, the stabilization effect of divalent stannous ion (Sn²⁺) in the oral care composition (toothpaste) was examined according to the presence of absence of stannous fluoride (SnF₂), stannous chloride (SnCl₂), and sodium gluconate. For the toothpastes prepared according to Example and Comparative Examples, the concentration of Sn²⁺ was examined after 4 weeks at 50 °C. The method of examining the concentration of Sn²⁺ is the same as the method performed in Experimental Example 1 above.

As a result of preparing the oral care compositions according to Example and Comparative Examples, the toothpaste of Example 2, in which both sodium gluconate and stannous chloride as stabilizers were added to the main ingredient stannous fluoride, showed the highest concentration of divalent stannous ions in the toothpaste. This means that the divalent stannous ions in the toothpaste were stabilized, and accordingly, the divalent stannous ions in the toothpaste may maintain their form even after 4 weeks of storage at 50 °C.

TABLE 3

| | Input of | Input of | | | | | | |
|---|---|---|---|---|---|---|---|---|
| | stannous | stannous | Input of | | | | | |
| | fluoride | chloride | sodium | Sn²⁺ ion concentration (wt %) | | | | |
| No. | (SnF₂) | (SnCl₂) | gluconate | Initial | 1 week | 2 weeks | 3 weeks | 4 weeks |
| Example 2 | ◯ | ◯ | ◯ | 0.628 | 0.378 | 0.326 | 0.289 | 0.303 |
| Comparative | ◯ | X | X | 0.344 | 0.234 | 0.177 | 0.136 | 0.15 |
| Example 4 | | | | | | | | |
| Comparative | ◯ | ◯ | X | 0.628 | 0.405 | 0.327 | 0.282 | 0.294 |
| Example 5 | | | | | | | | |
| Comparative | ◯ | X | ◯ | 0.344 | 0.226 | 0.189 | 0.152 | 0.16 |
| Example 6 | | | | | | | | |

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

1. An oral care composition comprising:
a zinc compound that is dissociated in an aqueous solvent to provide zinc ions; and
a stannous compound of which at least part is provided in the form of stannous gluconate,
wherein the stannous compound comprises stannous chloride (SnCl₂) and stannous fluoride (SnF₂).

2. The oral care composition of claim 1, wherein the zinc compound has a solubility of more than 0.6% and less than 69% in the aqueous solvent.

3. The oral care composition of claim 1, wherein the zinc compound is one or more selected from zinc citrate, zinc lactate, and zinc acetate.

4. The oral care composition of claim 1, further comprising a pH adjuster and an abrasive.

5. The oral care composition of claim 1, wherein pH of the oral care composition is 6 to 10.

6. A method of preparing an oral care composition, the method comprising:
a first step of forming stannous gluconate by introducing stannous chloride (SnCl₂) to a solution in which sodium gluconate is dissolved;
a second step of stabilizing stannous fluoride (SnF₂) in the form of stannous gluconate by adding stannous fluoride (SnF₂) to the mixed solution comprising stannous gluconate; and
a third step of adding the solution comprising stannous fluoride of the second step to a zinc compound solution comprising dissociated zinc ions.

7. The method of claim 6, wherein the pH adjuster is further added to the mixed solution after the third step.

8. The method of claim 6, wherein the zinc compound solution is prepared by dissolving a zinc compound having a solubility of more than 0.68 and less than 69% in an aqueous solvent.
